(19) United States
(12) Patent Application Publication
(10) Pub. No.: US 2022/0079784 A1
(43) Pub. Date: Mar. 17, 2022

(54) STENTS AND METHODS FOR USE AND MANUFACTURE OF STENTS WITH IMPROVED RETENTION MEMBERS

(71) Applicant: Boston Scientific Scimed, Inc., Maple Grove, MN (US)

(72) Inventor: Martyn G. Folan, Galway (IE)

(21) Appl. No.: 17/473,438

(22) Filed: Sep. 13, 2021

Related U.S. Application Data
(60) Provisional application No. 63/078,019, filed on Sep. 14, 2020.

(51) Int. Cl. A61F 2/86

(52) U.S. Cl. CPC A61F 2/86; A61F 2210/0014

(57) ABSTRACT

The present disclosure relates generally to the field of medical devices. In particular, the present disclosure relates to medical devices for facilitating the flow of fluids and materials in a lumen and/or between adjacent body lumens; for example, a stent which maintains an open flow passage between body lumens. In one example, a stent may comprise first and second flanges and an elongate body therebetween. The elongate body may extend outwardly beyond the elongate body such that ends may be doubled back into the respective first and second flanges.

### PRIORITY

The present application is a non-provisional of, and claims the benefit of priority under 35 U.S.C. § 119 to, U.S. Provisional Application Ser. No. 63/078,019, filed Sep. 14, 2020, the disclosures of which are herein incorporated herein by reference in their entirety for all purposes.

### FIELD

The present disclosure relates generally to the field of medical devices. In particular, the present disclosure relates to medical devices for facilitating the flow of fluids and materials, such as a stent which maintains an open flow passage between or within body lumens.

### BACKGROUND

Placement of a self-expanding metal stent (SEMS) within an anatomical area (e.g., body lumen, passage, vessel, duct, etc.) may enable fluid communication from one area to another. For example, a stent may enable flow of material from one body lumen to another.

However, available stents may carry various risks for a patient. For example, stents may migrate, such as in response to forces generated by a patient's motion. Additionally, stents may contribute to injury beyond the procedure. Post-acute bleeding, for example, may contribute to pain and infection risk for patients through abrasions and the exposure thereof to potentially dangerous material, such as digestive fluids or necrotic tissue. Stent designs with flanges having an interior open to the stent lumen may retain undesired biological tissue or other material which may provide a platform for infection.

With the above considerations in mind, a variety of advantageous medical outcomes may be realized by the devices and/or methods of the present disclosure.

### SUMMARY

In one aspect, a stent may comprise an elongate body, a first flange, a second flange, and a central portion therebetween formed from the elongate body in an expanded configuration. Ends of the elongate body may be doubled back into the respective first and second flanges.

In the described and other aspects of the present disclosure, the elongate body may be formed across an inner opening of the first and second flanges. The stent may comprise an unexpanded configuration when constrained by a delivery device prior to being deployed. A diameter of the first flange, the second flange, or both, may be larger than a diameter of the elongate body in the expanded configuration. The ends may be doubled back entirely into the respective first and second flanges such that respective ends of the stent comprise flush end faces. The doubled back ends of the elongate body may extend beyond one or both of the respective first and second flanges along the elongate body towards the opposing flange. The doubled back ends of the elongate body extend beyond one or both of the respective first and second flanges to form at least one additional flange adjacent to one or both of the respective first and second flanges. The doubled back ends of the elongate body may extend beyond one or both of the respective first and second flanges to overlap with an opposing doubled back end of the elongate body. At least the first flange and the second flange may be formed of a braided wire. The first flange, the second flange, or both, may comprise a coating. The elongate body may comprise a coating. The coating may constrain at least one doubled back end of the elongate body along a radially outward surface of the elongate body. The coating may comprise silicone. The elongate body may be self-expanding and comprises a shape-memory material. A valve may be disposed within the lumen.

In one aspect, a method may comprise forming an elongate body around a central mandrel. The elongate body may comprise a first portion, a second portion, and a cylindrical saddle region extending therebetween in an expanded configuration. The method may comprise forming at least one retention member by one or both of: doubling back the first portion over a first flange forming washer to form a first retention member, and doubling back the second portion over a second flange forming washer to form a second retention member.

In the described and other methods of the present disclosure, the elongate body may comprise a shape-memory material. The method may further comprise heat setting the expanded configuration for the stent having the at least one retention member. The method may further comprise covering at least part of the elongate body with a coating. The coating may constrain a first termination of the first portion, or the coating constrains a second termination of the second portion, or both.

In another aspect, a stent may comprise a stent body having a constrained configuration and an expanded configuration. The stent body may comprise a tubular structure including a first portion, a second portion, and a central portion extending therebetween in the expanded configuration. One or both of the first portion and the second portion may be doubled back toward the opposing portion to form, respectively, a first flange and second flange.

In the described and other aspects of the present disclosure, the stent body may be formed across an inner opening of the first and second flanges. The doubled back ends of the tubular structure may extend beyond one or both of the respective first and second flanges along the tubular structure towards the opposing flange. The ends may be doubled back entirely into the respective first and second flanges such that respective ends of the stent comprise flush end faces. The doubled back ends of the tubular structure may extend beyond one or both of the respective first and second flanges along the tubular structure towards the opposing flange. The stent may comprise a coating. The coating may constrain at least one doubled back end of the tubular structure along a radially outward surface of the tubular structure.

### BRIEF DESCRIPTION OF THE DRAWINGS

Non-limiting examples of the present disclosure are described with reference to the accompanying figures, which are schematic and not intended to be drawn to scale. In the figures, each identical or nearly identical component illustrated is typically represented by a single numeral. For purposes of clarity, not every component is labeled in every figure, nor is every component in each embodiment of the disclosure shown where illustration is not necessary to allow those of skill in the art to understand the disclosure. In the figures:

FIGS. 1A-1B illustrate side views of a self-expanding stent, with FIG. 1A depicting the stent disposed in tissue spanning two body lumens.

FIG. 2A illustrates an elongated body of a self-expanding stent, according to one embodiment of the present disclosure.

FIG. 2B illustrates a cross-sectional side view of a self-expanding stent, according to one embodiment of the present disclosure.

FIG. 3 illustrates a cross-sectional side view a self-expanding stent with components for the manufacture thereof, according to one embodiment of the present disclosure.

FIGS. 4A-4H illustrate cross-sectional side views of self-expanding stents, according to embodiments of the present disclosure.

FIG. 5 illustrates a cross-sectional side view of a self-expanding stent disposed in tissue spanning two body lumens, according to one embodiment of the present disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices and systems for drainage, e.g., of pancreatic fluid collections, it should be appreciated that such medical devices may be used to establish and/or maintain a temporary or permanent open flow passage between or within a variety of body organs, lumens and spaces, e.g., the stomach, duodenum, kidneys, and/or gall bladder, as well as the gastrointestinal, respiratory, urinary and/or reproductive tracts. For example, such medical devices may be used to provide gastroduodenal or gastrojejunal bypasses, anastomoses between two anatomical regions, biliary drainage, gallbladder drainage, treatment of walled off necrosis (WON), or other uses. The devices can be inserted via different access points and approaches, e.g., percutaneously, endoscopically, laparoscopically or some combination. Various stents described are self-expanding, but other embodiments where the stent is expandable by other means, for example, a balloon catheter, may be possible. Moreover, such medical devices are not limited to drainage, but may facilitate access to organs for other purposes, such as removing or relieving obstructions and/or delivering therapy, including non-invasive or minimally invasive manipulation of the tissue within the organ and/or the introduction of pharmacological agents via the open flow passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

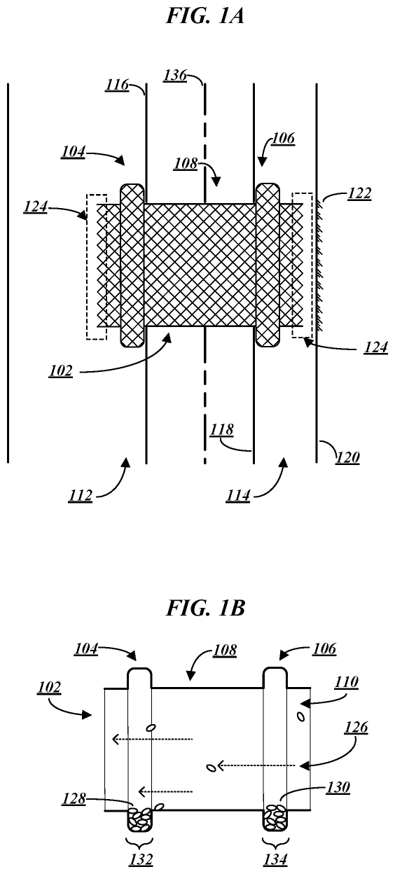

Referring to FIGS. 1A-1B, a stent 102 may comprise retention members such as flange 104 and flange 106, with an elongated body 108 extending therebetween. Although an uncovered stent is illustrated for the sake of clarity, it is understood that the stent 102 may be uncovered, partially covered, or fully covered. The stent 102 may be disposed in an anatomical body, for example, providing a conduit between body lumen 112 and body lumen 114 through a single layer or bridging multiple tissue layers. For example, the flange 104 may be configured to contact a first tissue wall 116 and a flange 106 may be configured to contact a second tissue wall 118. The first tissue wall 116 and the second tissue wall 118 may be walls of the same or of different tissue layers or body lumens. For example, a first flange and a second flange may contact a first tissue wall of a first tissue and a second tissue wall of a second tissue so that the first tissue and the second tissue come into contact with each other or otherwise interact at a tissue interface, for example, at tissue interface 136. In some embodiments, at least one additional tissue layer may be positioned between a tissue layer of the first tissue wall 116 and a tissue layer of the second tissue wall 118 (not illustrated). However, the stent 102 may wear on an adjacent tissue wall 120, for example, due to a restricted area of a body lumen, such as the body lumen 114. Abrasions 122 may form on the adjacent tissue wall 120 as a result of interaction with a stent end 124. For example, the stent end 124 may include one or more terminations of a braided structure or other material, which may be pointed and cause abrasions on an adjacent tissue wall. The stent end 124 may refer to one or both of a first or a second end of a stent.

FIG. 1B displays a cross section of a stent, such as the stent 102, with a lumen 110 extending laterally therethrough. For the sake of clarity in the drawings, the cross-sectional drawings herein include an outline rather than an illustration of each of the wires illustrated in FIG. 1A, but it will be understood that the stent of FIG. 1B and/or any of the stents herein may comprise a mesh structure of a woven wire or wires, such as illustrated in FIG. 1A. Furthermore, while vertical contour lines are included in the cross-sectional drawing of FIG. 1B, further cross-sectional drawings of the present disclosure will not show vertical contour lines in order to allow clear interpretation of other features. As shown by the cross section of FIG. 1B, a cavity or gulley of a first flange and/or a second flange may provide a reservoir into which fluid and/or material may flow and potentially be retained, for example, reservoir 132 and/or reservoir 134. For example, flow 126, indicated by dotted arrows, may include necrotic material, which may be solid or semi-solid. As the flow 126 proceeds through the lumen 110, fluid and/or other material may result in accumulation of the flow 126 content in a reservoir of the stent, such as reservoir 132 and/or reservoir 134. For example, solid particles suspended in a fluid of the flow 126 may be caught in an accumulation 128 and/or an accumulation 130. It is understood that orientation of the stent 102 in a patient may affect accumulation of flow 126 in reservoirs 132, 134 of flange 104, 106. Flow and/or contents thereof may be accumulated in accordance with a directionality of the flow. For example, accumulation may build up more in a downstream flange reservoir than in an upstream flange reservoir according to decreased turbulence within the flow at the downstream end of the stent. Alternatively, or additionally, flow accumulation may be affected by other forces, such as gravity. For example, flow may accumulate more in a lower flange reservoir of a vertically oriented stent than in an upper flange reservoir as an effect of gravity. In some cases, the accumulation 128 and/or the accumulation 130 may build to restrict the passage of the flow 126 through the lumen 110. In some cases, the accumulation 128 and/or the accumulation 130 may provide a loci for infection and related inflammation.

In some embodiments, the stent 102 disposed in an anatomical region associated with high stresses may migrate or may not be able to maintain a fully open lumen due to surrounding forces. For example, a muscular tissue may exert a force on the stent 102 great enough to dislodge the stent 102 from a proper position in an anatomical region. In another example, a disposition of the stent 102 in a tissue with high muscular mass (e.g., the gastric wall) may not be able to maintain a complete expansion of the elongated body 108.

These considerations, among others, may be addressed by the present disclosure. Embodiments described herein may, for example, enable flow between body lumens without exposing surrounding tissue to abrasive device configurations and/or without creating reservoirs for potentially infectious material.

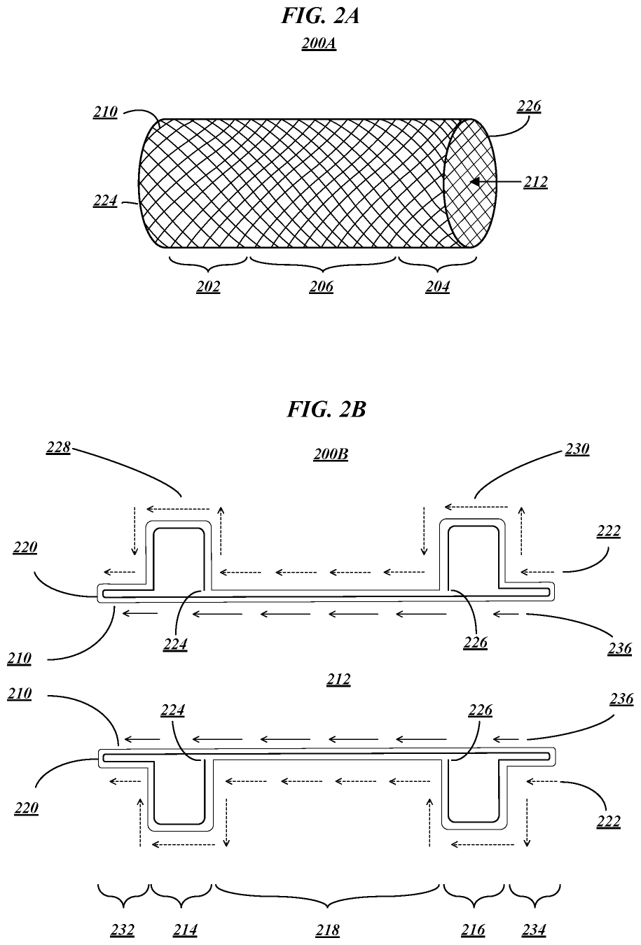

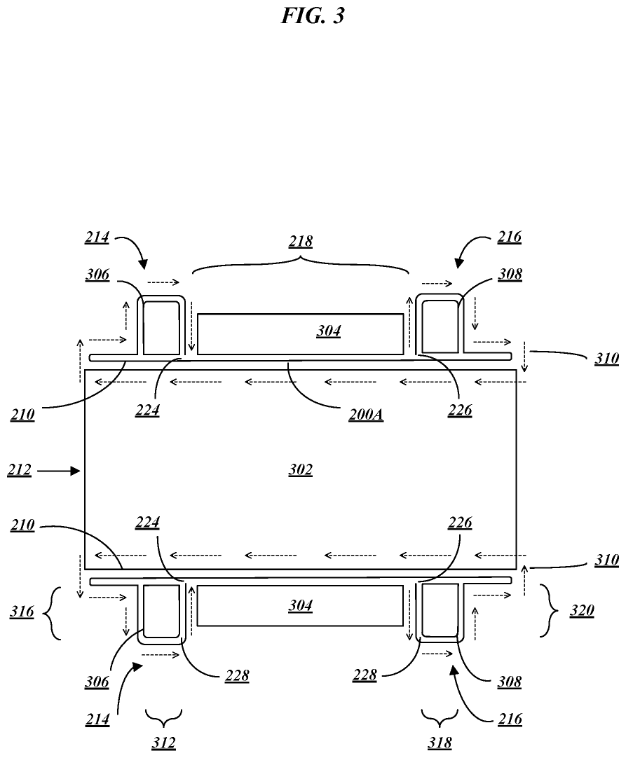

FIGS. 2A-2B illustrate aspects of manufacturing a stent configuration according to an embodiment of the present disclosure. A stent may be formed from an elongated body of stent material, such as elongated body 200A, a tubular structure, a cylindrical mesh member, or the like. The elongated body 200A may be formed on a central mandrel, as described below with respect to FIG. 3, or it may be pre-formed and subsequently loaded onto a central mandrel. Then, washers and methods as described with respect to FIG. 3 may be used to form a completed stent structure according to one or more embodiments herein, such as stent 200B of FIG. 2. While a stent may be formed according to various embodiments described herein using alternative tubular structures, elongated body 200A is illustrated separately in FIG. 2A by way of example in order to provide context for completed stent constructions according to methods described below.

In many embodiments, an elongated body 200A may include a wire, mesh, or other frame known in the art, such as braided wire 210. According to many embodiments, a stent may be formed using either a single wire or multiple wires, although other types and arrangements of filaments or wires may be used in various configurations. For example, such filaments or wires, whether as a single wire or multiple wires, may be configured as a woven, braided, or knitted mesh, or the like. In some embodiments, the elongated body 200A may include a length of wire braid as described below. A frame material such as the braided wire 210 may form a cylindrical tube including a first portion 202, a second portion 204, and a central portion 206 extending therebetween. For example, a first portion corresponding to a first flange, a second portion corresponding to a second flange, a central portion corresponding to a cylindrical saddle region, or any combination thereof, may be formed of a braided wire. The length of each of the first portion 202, second portion 204, and central portion 206 may differ between embodiments to provide sufficient length for various flange dimensions, cylindrical saddle region dimensions, protruding end dimensions, saddle interactions, and other components as described herein.

An elongate body may define a lumen extending therethrough. For example, the first portion 202, second portion 204, and central portion 206 may define a lumen 212 extending longitudinally therethrough. The lumen 212 may include various dimensions suited for different purposes, as described below. At the end of an elongated body, material may be connected. For example, wires may be welded or otherwise joined together so that a closed, braided structure may be maintained. Accordingly, a first end of the elongated body 200A may end in one or more first welded terminations 224, a second end of the elongated body 200A may end in one or more second welded terminations 226, or both. In some embodiments, one end may include bent portions of wires, with wire ends being woven and terminating at the other end, where they may be joined. While welded terminations are referred to for the sake of simplicity, it will be understood that one or more aspects of the disclosure may include glued, twisted, or otherwise joined terminations of wire.

An elongated body 200A may be formed into a stent 200B, a cross sectional view of which is illustrated in FIG. 2B. In many embodiments, a stent may comprise a constrained configuration (e.g., unexpanded configuration) and an unconstrained configuration (e.g., expanded configuration). An elongate body may extend beyond one or both of first and/or second flanges to extend along the elongate body towards the opposing flange. For example, the first portion 202 of a stent may be doubled back or folded back along an outer surface of the central portion 206 so as to extend towards the second portion 204. The first portion 202 may be doubled back to form a retention member, for example, a first flange 214, around an outer diameter of the central portion 206. Accordingly, the first welded termination 224 of the elongate body 200A may no longer be located at a first end of the stent structure, but wrapped away from a first protruding end 232, or lip, which may form the first end of the stent 200B. The first protruding end may be formed from an excess length of first portion 202 folded back to form first flange 214. Likewise, the second portion 204 of a stent may be doubled back, for example, along an outer surface of the central portion 206 so as to extend towards the first portion 202. The second portion 204 may be doubled back to form a retention member, such as second flange 216, around an outer diameter of the central portion 206. The first flange 214 and/or a second flange 216 may comprise a double-walled structure extending radially outward from the central portion 206. The second welded termination 226 of the elongate body 200A may no longer be located at a second end of the stent structure, but wrapped away from a second protruding end 234, or lip, which may form the second end of the stent 200B. In other words, one or more doubled back ends of an elongate body may extend beyond one or both of the respective first and second flanges along the elongate body towards the opposing end or opposing portion, which may form or otherwise include an opposing flange or other retention member. The second protruding end may be formed from an excess length of second portion 204 folded back to form first flange 214. That is, the elongate body 200A may extend axially outwardly beyond first and/or second retention members, e.g., flanges, such that respective ends are doubled back into the respective first and/or second flanges.

A cylindrical saddle region 218, comprising an elongated, tubular structure, may extend between the first flange 214 and the second flange 216. When referring to the “saddle region” of a stent, the region may refer to the portion of the stent between two retention members (e.g., flanges) in a deployed configuration. Depending on how the retention members are arranged, this portion may coincide with the overall length of the stent in the deployed configuration if the stent ends coincide with the position of the retention members. In other embodiments, the retention members may overhang the body of the stent, in which case the saddle region may refer to the portion of the stent between the beginning of the retention members where they meet the body of the stent, or the saddle region may be considered the portion of the body of the stent between the retention members at a point along the retention members where they are closest to each other. In some embodiments, a length of a saddle region may be measured for a deployed configuration that represents the configuration when deployed off of the delivery member, but not in tissue, and in other cases represents the configuration when deployed in or across tissue. Such lengths may be the same or differ.

In some embodiments, a length of a saddle region may correspond to a length of a central portion. For example, the length of the cylindrical saddle region 218 may correspond to a length of the central portion 206, which may extend between first and second portions 202, 204 folded to form respective flanges as described with respect to FIG. 2A. It will be recognized that a first protruding end and/or a second protruding end may be less abrasive than a corresponding stent end with at least one welded termination, for example, the stent end 124 as described with respect to FIG. 1A, or a corresponding stent end that has no welded terminations.

A flange may define or otherwise encompass a cavity, or reservoir. For example, first flange 214 may define a cavity 228 and second flange 216 may define a cavity 230. In many embodiments, the cavity may be empty, enabling a collapse of the flange when sufficient pressure is applied to an outer surface of the first flange. For example, the first flange 214 may be collapsed and/or elongated and the second flange 216 may be collapsed and/or elongated when the stent 200B is in a constrained configuration.

As an advantage of a doubled back design of a stent, such as stent 200B, a cavity such as cavity 228 and/or cavity 230 may be separated from a lumen through which fluid and/or other materials may flow. A wall of elongate body 200A may separate the cavity 228 and the cavity 230 from the lumen 212. For example, an elongate body may be formed across an inner opening of first and/or second retention members. In many embodiments, at least a radially interior wall defining the lumen may be coated or covered, resulting in a covering or coating further separating a cavity from the lumen. In many embodiments, flow from the lumen may accordingly be minimized or prevented from entering the cavity 228. For example, flow through the lumen 212 may follow the directionality of solid arrows 236. Accordingly, the saddle region 218 separating the lumen 212 from cavity 228 and/or cavity 230 may inhibit or prevent an accumulation of material carried by flow through the lumen 212, and thereby may reduce risk for occlusion of the lumen 212, reducing potential loci for a gathering of infectious material, or both.

In many embodiments, a stent, such as stent 200B, may include a covering or coating, such as cover 220. In some embodiments, all or a portion of an elongate body, a first flange, a second flange, a cylindrical saddle region, or any combination thereof may comprise a coating or a cover. A cover 220 is illustrated as an outer and inner coating for the sake of clarity in FIG. 2B, but it will be recognized that a cover may be a partial or a full covering for a stent. A cover may be applied by dip coating, hand painting, spraying, or other known deposition technique. For example, the cover 220 may be applied in a defined directionality, such as in accordance with dashed arrows 222, which may or may not correspond with a directionality of a material used to define the stent 200B, such as the braided wire 210. In many embodiments, an inner surface of the stent 200B, such as defining the lumen 212, may comprise a cover. For example, stent 200B may be fully or partially encapsulated by or positioned within a cover 220.

A cover or coating may extend over at least one of the first flange 214 or the second flange 216. In many embodiments, a cover or coating may extend over at least one of a first welded termination or a second welded termination. In many embodiments, the cover or coating constrains at least one termination of the elongate body along a radially outward surface of the elongate body. Accordingly, a cover may act as a binding or securing agent to hold a first portion and/or a second portion, for example, by a first welded termination or a second welded termination, in a location with respect to a wall of the central portion of the elongated body. Specifically, a cover may hold a first welded termination or a second welded termination in a location to define a shape and/or dimension of a first flange, a second flange, a cylindrical saddle region, or any relevant combination thereof. For example, the cover 220 may hold the first welded termination 224 or the second welded termination 226 in a location to define a shape and/or dimension of the first flange 214, the second flange 216, the cylindrical saddle region 218, or any combination thereof.

In many embodiments, a flange formed by doubling back a portion of an elongate body, for example, the first flange 214 or second flange 216, as described with respect to FIG. 1B, may have an increased rigidity, in terms of flexibility, pull-out or retentive force, and/or radial force, compared to a corresponding flange formed without a doubled back portion of the elongate body, such as the flange 104 or the flange 106 as described with respect to FIG. 1B, respectively. The securement of a respective wire termination, such as a welded, glued, twisted, or otherwise joined termination, by the cover 220 may further increase the strength of the first flange 214, the second flange 216, or both, in terms of one or more of the forces described above. For example, securement of a wire termination may inhibit or prevent a respective flange from freely deforming, compared to a configuration without such securement, when a force (e.g., pull-out force) is applied to the flange along a longitudinal axis of a cylindrical saddle region. Accordingly, a securement of a wire termination may result in a greater force required to dislodge a stent with a doubled back flange configuration from a lumen (i.e., pull out force) than to dislodge a stent with an alternative flange configuration, such as flange 104 or flange 106.

In many embodiments, the cover 220 may extend over the first flange 214 and/or the first welded termination 224 so as to close or substantially close off the cavity 228 from an anatomical environment, reducing risk for the access of the cavity by biological material and an associated subsequent risk for an infection thereof. Similarly, the cover 220 may extend over the second flange 216 and/or the second welded termination 226 so as to close or substantially close off the cavity 230 from an anatomical environment.

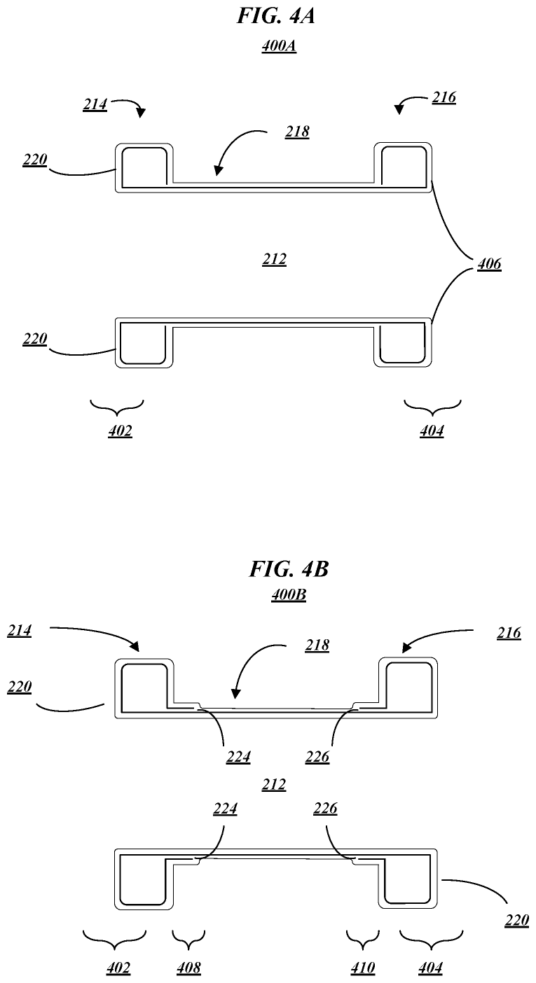

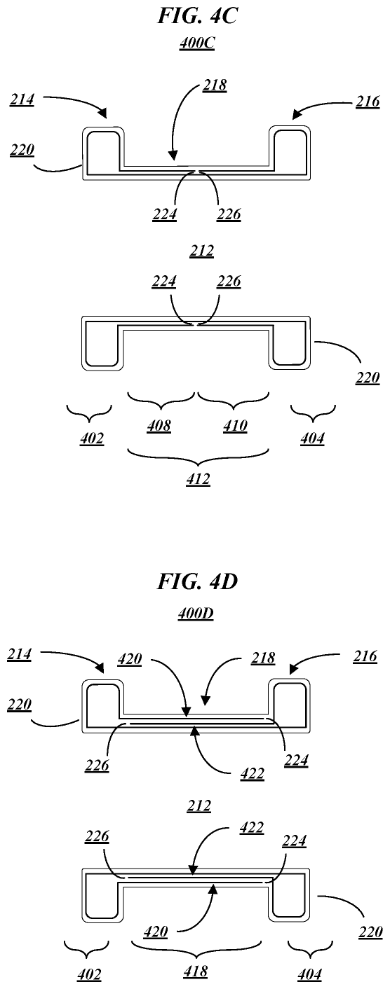

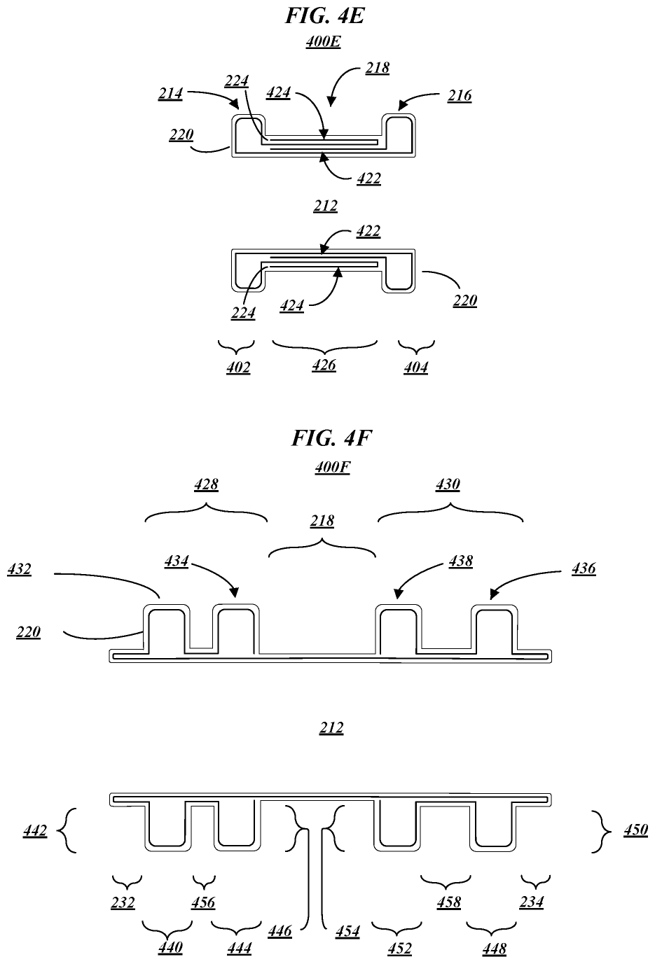

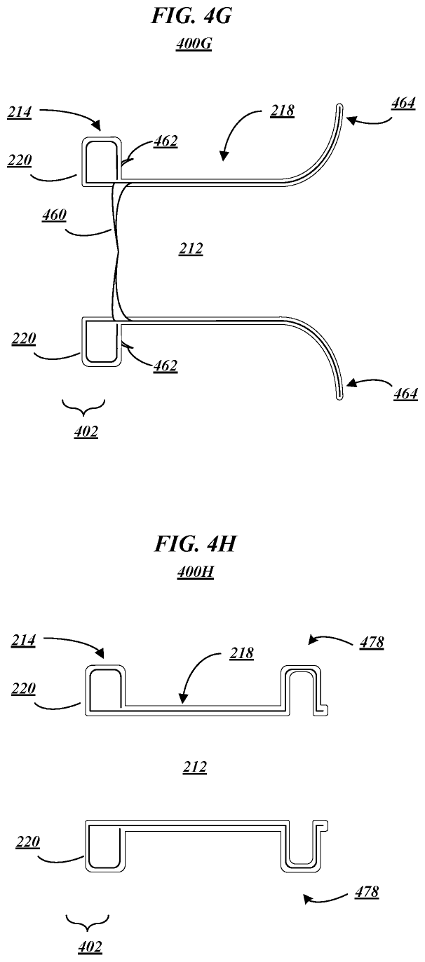

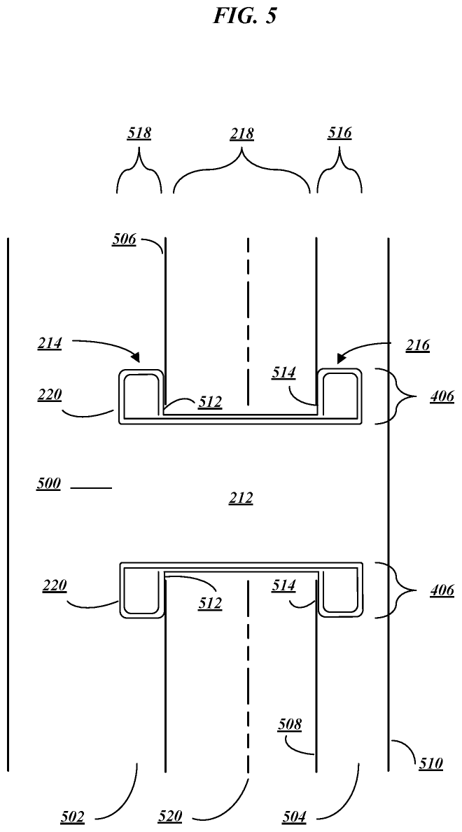

FIG. 3 illustrates exemplary aspects of a method for manufacturing a stent according to one or more embodiments herein. For example, the stent 200B as described with respect to FIG. 2B may be manufactured using components described with respect to FIG. 3. For the sake of simplicity, reference will be made in FIG. 3 to one or more components as described with respect to FIG. 2A-2B. However, embodiments are not limited in this context. For example, components and/or methods described with respect to FIG. 3 may be used to manufacture embodiments described with respect to FIGS. 4-5.

An elongated body, such as elongated body 200A, may be formed around a central mandrel, such as central mandrel 302. The central mandrel may accordingly set an inner diameter of a lumen extending through the elongated body. In many embodiments, the central mandrel may be cylindrical. A saddle clip, such as saddle clip 304, may constrain an outer diameter of the elongated body. Specifically, a saddle clip may extend partially or entirely around a circumference of an elongated body, constraining and thereby defining an outer diameter of a cylindrical saddle region.

In many embodiments, a first flange forming washer and/or a second flange forming washer, such as respective washers 306, 308, may be placed or otherwise located around an outer circumference of the elongated body. In many embodiments, a first flange forming washer and a second flange forming washer may have similar or the same dimensions. In other embodiments, dimensions of a first flange forming washer and a second flange forming washer may differ.

A portion of an elongated body may be doubled back over a flange forming washer so that a width and diameter of the flange forming washer may define an inner width and/or an inner diameter of a cavity of a flange. For example, first portion 202, as described with respect to FIG. 2A, of the elongated body 200A may be doubled back over the first flange forming washer 306 so that a width 312 of the first flange forming washer 306 may define an inner width of a cavity 228. Likewise, a diameter 316 of the first flange forming washer 306 may define an inner diameter of the cavity 228. Similarly, a second portion, such as the second portion 204 as described with respect to FIG. 2A, of the elongated body 200A, may be doubled back over the second flange forming washer 308 so that a width 318 of the second flange forming washer 308 may define an inner width of a cavity 230. Likewise, a diameter 320 of the second flange forming washer 308 may define an inner diameter of the cavity 230. Accordingly, the width and the diameter of a flange may be determined by the corresponding dimensions of the flange forming washer. A flange formed by doubling back a portion of an elongate body over a flange forming washer may comprise a diameter larger than a diameter of the elongate body. A diameter of a first flange and/or a second flange may be set to have a particular offset from an outer diameter of a cylindrical saddle region, as described below.

In many embodiments, an elongated body, such as elongated body 200A, may comprise a braided wire structure. The wire structure may follow, for example, a wire path as illustrated by dashed arrows 310. For example, after the first portion and the second portion of the elongated body 200A have been doubled back and formed into retention members, such as discussed above, a wire length may extend from the second welded termination 226 around the second flange 216, along the cylindrical saddle region 218, and around the first flange 214, ending in the first welded termination 224. Embodiments are not limited in this context.

After an elongated body has been doubled back as described above, the shape of the wire may be set, for example, in an expanded configuration (e.g., unconstrained configuration) of a self-expanding body. For example, the elongated body 200A and/or braided wire may comprise a shape-memory material, such as Nitinol or a similar alloy. The shape may be set, for example, by heat-setting. After the shape of the stent structure has been set, the central mandrel, saddle clip, first flange forming washer and/or second flange forming washer may be removed.

In many embodiments, a covering, coating, or other layer may be applied to the stent structure. For example, cover 220 may be applied, as described with respect to FIG. 2B. A cover may comprise, for example, silicone, other polymer material, or a combination thereof. For example, a cover may comprise polyurethane, polytetrafluoroethylene, polyvinylidene fluoride, an aromatic polycarbonate-based thermoplastic urethane, and/or another material, or the like. A cover may be applied by dip coating, painting, spraying, other known deposition method, or a combination thereof.

FIGS. 4A-G illustrate cross-sectional views of various stent configurations according to one or more embodiments described herein. Components may be referenced therein which are similar to one or more components described with respect to FIG. 2A-B and FIG. 3. Embodiments are not limited in this context.

In a configuration as illustrated by FIG. 4A, a stent 400A may comprise a first retention member, a second retention member, a cylindrical saddle region, and a lumen. For example, components of stent 400A may respectively comprise one or more similar aspects of the first flange 214, second flange 216, cylindrical saddle region 218, and lumen 212 as described with respect to FIG. 2B. However, rather than including the first protruding end 232 and the second protruding end 234 as does stent 200B, stent 400A may comprise a first flush end face 402 and/or a second flush end face 404. A first flush end face 402 may be manufactured using aspects described with respect to FIG. 3, but wherein the first flange forming washer 306 is positioned to be flush or near flush with the intended end of the stent. In other words, a shorter first portion of the elongate body 200A may be doubled back that does not include wire length to form the first protruding end 232 adjacent to the formed first flange 214. Similarly, the second flush end face 404 may be manufactured using aspects described with respect to FIG. 3, but wherein the second flange forming washer 308 is positioned to be flush or near flush with the respective intended end of the stent. In other words, a shorter second portion of the elongate body 200A may be doubled back that does not include wire length to form the second protruding end 234 adjacent to the formed second flange 216. Alternatively, or additionally, ends may be doubled back entirely into respective first and/or second retention members such that the respective end of the stent comprises a flush end face.

Flush end faces may present lower risk of abrasion of adjacent tissue by the end of the stent. For example, an adjacent tissue coming into contact with a second end of the stent may interact with a sufficiently flat covered side 406 of the second flange 216 rather than, for example, the stent end 124 containing wire terminations as described with respect to FIG. 1.

In some embodiments, such as FIG. 4A, a stent 400A may comprise a cover, such as cover 220 described with respect to FIG. 2B.

In a configuration as illustrated by FIG. 4B, a stent 400B may comprise a first retention member, second retention member, cylindrical saddle region, and lumen. For example, the stent 400B may comprise the first flange 214, second flange 216, cylindrical saddle region 218, and/or lumen 212 as described with respect to FIG. 2B. Stent 400B is illustrated with the first flush end face 402 and second flush end face 404 as described with respect to FIG. 4A, but it will be recognized that stent 400B may alternatively include at least one alternative end configuration, such as a first protruding end 232 or a second protruding end 234 as described with respect to FIG. 2B.

Embodiments such as stent 400B may comprise a partial first saddle interaction and/or a partial second saddle interaction. A partial first saddle interaction, such as partial first saddle interaction 408, may be formed at least in part according to aspects discussed with respect to FIG. 3. Specifically, a partial first saddle interaction may be formed by folding a first portion of an elongate body to extend along a cylindrical saddle region towards the second portion beyond a first flange. Accordingly, a first welded termination may lie parallel to the cylindrical saddle region. The length of the partial first saddle interaction may be the distance by which a first portion is doubled back to extend along a cylindrical saddle region beyond a first flange. Similarly, a partial second saddle interaction, such as partial second saddle interaction 410, may be formed by folding a second portion of an elongate body to extend along a cylindrical saddle region towards the first portion beyond a second flange. Accordingly, a second welded termination may lie substantially parallel to a cylindrical saddle region. The length of a partial second saddle interaction may be the distance by which a second portion is doubled back to extend along a cylindrical saddle region beyond a second flange. In many embodiments, the stent 400B may be manufactured according to aspects discussed with respect to FIG. 3, wherein an elongate body extends beyond one or both of first and second flanges to overlap with an alternative termination of the elongate body. For example, a first portion of an elongate body may be doubled back over and beyond a first flange forming washer, such as first flange forming washer 306, such that the first portion lies against, overlaps with, or otherwise interacts with a termination of a second portion.

For example, with respect to stent 400B, the partial first saddle interaction 408 is formed, wherein a second portion of an elongate body is doubled back over and beyond the second flange forming washer 308 such that the partial second saddle interaction 410 is formed, or both. Then, the saddle clip 304 may be applied over the cylindrical saddle region 218, the partial first saddle interaction 408, the partial second saddle interaction 410, or any combination thereof, constraining the first welded termination 224 and/or the second welded termination 226 to lie against the outer surface of the cylindrical saddle region 218.

The cover 220 may be applied over at least the cylindrical saddle region 218 with the partial first saddle interaction 408 and/or the partial second saddle interaction 410 so that the cover may constrain or otherwise bind the partial first saddle interaction 408 and/or the partial second saddle interaction 410 to lie against the cylindrical saddle region 218 subsequent to the removal of the saddle clip 304.

An advantage of a partial first saddle interaction and/or a partial second saddle interaction may include an increased rigidity of a stent. In addition to the frame of a cylindrical saddle region, a partial first saddle interaction and/or partial second saddle interaction may resist a radially inward compressive force from a tissue for its respective length. Accordingly, the combination of the frame of the cylindrical saddle region 218 and the partial first saddle interaction 408 and/or the partial second saddle interaction 410 in an overlapping region may resist a greater radially compressive force than a corresponding section of a frame of a cylindrical saddle region 218 without a partial first saddle interaction 408 and/or a partial second saddle interaction 410, such as in stent 102 as described with respect to FIG. 1B. In many embodiments, an ability of a stent to withstand a radially inward compressive force along a cylindrical saddle region may be increased linearly with respect to an increase of a length of a partial first saddle interaction and/or an increase of a length of a partial second saddle interaction.

In some embodiments, a flexibility of a stent may be decreased along a length of a partial first saddle interaction and/or a partial second saddle interaction. For example, the flexibility of a stent may be linearly decreased over a portion of the cylindrical saddle region with each additional layer of doubled back stent body presented by a partial first saddle interaction and/or a partial second saddle interaction.

In many embodiments, a partial first saddle interaction and a partial second saddle interaction may be configured to have the same length, thereby providing each end of a cylindrical saddle region with symmetric radial rigidity. However, in some embodiments, one of a partial first saddle interaction or a partial second saddle interaction may be longer than the other, or a stent may include only one of a partial first saddle interaction or a partial second saddle interaction. A partial first saddle interaction may extend across an entire length of a cylindrical saddle region and be doubled back to extend along part of or all of a second flange in order to provide additional support to the second flange (not shown). Additionally, or alternatively, a partial second saddle interaction may extend across an entire length of a cylindrical saddle region and be doubled back to extend along part of or all of a first flange in order to provide additional support to the first flange (not shown). Accordingly, a stent may be configured to have asymmetrical radial strength between a first end and a second end. A stent with an asymmetrical radial strength may be useful, for example, in implantations in tissue with asymmetrical muscular mass, density, or other source of force. For example, applications of a stent in esophageal, tracheal, or gastroduodenal procedures may require engagement with tissues with varying characteristics.

In various embodiments, a partial first saddle interaction and/or a partial second saddle interaction may alternatively, or additionally, contribute to a stent having a higher pull out force than a respective stent without a partial first saddle interaction and/or a partial second saddle interaction. For example, the partial first saddle interaction and/or partial second saddle interaction may result in at least one terminal end and/or portion of wire extending into a terminal end being held or constrained in a position with respect to a cylindrical saddle region. The securement of at least one terminal end and/or portion of wire extending into a terminal end may restrict deformation of a respective flange in response to an applied axial force (e.g., pull-out force). Accordingly, a greater force may be required to dislodge the flange and/or stent from a tissue.

In a configuration as illustrated by FIG. 4C, a stent 400C may comprise a first retention member, second retention member, cylindrical saddle region, and lumen. For example, stent 400C may comprise one or more of the first flange 214, second flange 216, cylindrical saddle region 218, and lumen 212 as described with respect to FIG. 2B. Stent 400C is illustrated with the first flush end face 402 and second flush end face 404, as described with respect to FIG. 4A, but it will be recognized that stent 400C may alternatively include at least one alternative end configuration, such as the first protruding end 232 or second protruding end 234 as described with respect to FIG. 2B.

A stent may have a complete saddle interaction, such as with stent 400C illustrated in FIG. 4C. A complete saddle interaction 412 may be formed by a combination of the partial first saddle interaction 408 and the partial second saddle interaction 410, as described with respect to FIG. 4B, such that the first welded termination 224 and the second welded termination 226 meet or are sufficiently close to each other along the cylindrical saddle region 218 (shown). In other embodiments, a complete saddle interaction may be formed by an extension of only a first portion of an elongate body beyond a first flange such that a first welded termination is located at or sufficiently near to a second flange (not shown). In further embodiments, a complete saddle interaction may be formed by an extension of only a second portion of an elongate body beyond a second flange such that a second welded termination is located at or sufficiently near to a first flange (not shown).

Similar to stent 400B of FIG. 4B, stent 400C may include a greater ability to withstand radially compressive forces along the length of its cylindrical saddle region 218 due to support from the complete saddle interaction 412. Manufacturing methods for stent 400C may be similar to manufacturing methods for stent 400B.

In a configuration as illustrated by FIG. 4D, a stent 400D may comprise a first retention member, second retention member, cylindrical saddle region, and lumen. For example, stent 400D may comprise one or more of the first flange 214, second flange 216, cylindrical saddle region 218, and lumen 212 as described with respect to FIG. 2B. Stent 400D is illustrated with the first flush end face 402 and the second flush end face 404, as described with respect to FIG. 4A, but it will be recognized that stent 400D may alternatively include at least one alternative end configuration, such as the first protruding end 232 or the second protruding end 234 as described with respect to FIG. 2B.

Stent 400D may comprise an overlap region 418 along part of or all of the cylindrical saddle region 218. The overlap region 418 may be formed using aspects of FIGS. 2-3, which for simplicity are not illustrated in FIG. 4D. Specifically, an overlap region may be formed when a first portion, such as the first portion 202 as described with respect to FIGS. 2A-B, is doubled back to form a first retention member such as first flange 214 and a first saddle interaction 420 and when a second portion, such as the second portion 204 as described with respect to FIGS. 2A-B, is doubled back to form a second retention member such as second flange 216 and a second saddle interaction 422, wherein the first saddle interaction 420 and the second saddle interaction 422 are long enough so as to overlap. An outer diameter of the overlap region 418 may be set by a saddle clip such as saddle clip 304, and all or part of the overlap region 418 may be covered by a cover, such as cover 220, as described above.

An overlap region may contribute to added ability to withstand radially compressive forces along the length of its cylindrical saddle region due to mechanical support from the first saddle interaction and the second saddle interaction.

In cases where greater radial strength is desired, an embodiment may include additional length and/or folding of the first portion and/or the second portion to form at least one additional respective first saddle interaction or second saddle interaction. For example, with respect to FIG. 4E, stent 400E includes the first saddle interaction 424 which includes an additional fold to extend back along a length of the cylindrical saddle region 218. Accordingly, the first welded termination 224 is directed towards and/or near to a first end of the stent. The first saddle interaction 424 and/or the second saddle interaction 422 may contribute to a region of overlapping portions of the elongate body 200A in an overlap region 426. The cover 220 may be applied over at least the overlap region 426. Stent 400E is illustrated with the second saddle interaction 422 as described with respect to FIG. 4D, but it will be understood that either the first saddle interaction 424 and/or the second saddle interaction 422 may include any number of doubled back layers. In some embodiments, the stent 400E may include only one of the first saddle interaction 424 or the second saddle interaction 422.

In many embodiments, stent 400E may include one or more of a first retention member, second retention member, cylindrical saddle region, and a lumen described above, such as first flange 214, second flange 216, cylindrical saddle region 218, and lumen 212. Stent 400E is illustrated with the first flush end face 402 and the second flush end face 404, as described with respect to FIG. 4A, but it will be recognized that stent 400E may alternatively include at least one alternative end configuration, such as the first protruding end 232 or the second protruding end 234 as described with respect to FIG. 2B.

A stent with a doubled back configuration may include one or more retention members with alternative configurations. In some embodiments, an elongate body may extend beyond one or both of first and second flanges to form at least one additional flange adjacent to the respective flange. For example, stent 400F of FIG. 4F includes a first double flange 428 as a first retention member and a second double flange 430 as a second retention member. The stent 400F may be constructed by using one or more aspects described above, which may or may not be illustrated in FIG. 400F for the sake of simplicity. For example, the stent 400F may include a cylindrical saddle region 218 and a lumen 212, as described above. The first double flange 428 may be formed by folding a first portion of an elongate body, such as first portion 202 described with respect to FIG. 2A, over multiple first flange forming washers or over a single flange forming washer, such as first flange forming washer 306, multiple times. For example, an additional first flange forming washer (not illustrated) may be placed near the first flange forming washer 306 along the elongated body 200A as described with respect to FIG. 3. A first portion of an elongated body, such as the first portion 202 of the elongated body 200A as described with respect to FIG. 2A, may be doubled back over the first flange forming washer 306 and the additional first flange forming washer to form a first exterior flange 432 and a first interior flange 434. Similarly, an additional flange forming washer (not illustrated) may be placed near the second flange forming washer 308 along the elongated body 200A as described with respect to FIG. 3. A second portion of an elongated body, such as the second portion 204 of the elongated body 200A as described with respect to FIG. 2A, may be doubled back over the second flange forming washer 308 and the additional flange forming washer to form a second exterior flange 436 and a second interior flange 438. In some examples, a single flange forming washer may be used to form a flange, then be moved axially along an elongate body and subsequently used to form an additional flange.

Various dimensions of flange forming washers may be used to create flanges of specific dimensions. For example, each of a width 440 of the first exterior flange 432, a width 444 of the first interior flange 434, a width 448 of the second exterior flange 436, and a width 452 of the second interior flange 438 may be the same or different. Each of a diameter 442 of the first exterior flange 432, a diameter 446 of the first interior flange 434, a diameter 450 of the second exterior flange 436, and a diameter 454 of the second interior flange 438 may be the same or different. Spacing between flanges of retention members may be the same or different in order to accommodate interaction with various tissue thicknesses. For example, first flange interspace 456 may be the same or a different width than second flange interspace 458.

Stent 400F is illustrated with the first protruding end 232 and the second protruding end 234, although it will be recognized that one or more ends of stent 400F may include an alternative end configuration, such as the first flush end face 402 or the second flush end face 404 as described above.

In various embodiments, at least one additional saddle clip, for example, an additional saddle clip to the saddle clip 304 described with respect to FIG. 3, may be used to define an outer diameter of one or more of the first flange interspace 456, the second flange interspace 458, the first protruding end 232, or the second protruding end 234.

A cover may be applied to all or part of stent 400F, such as cover 220 as described above with respect to FIG. 2B.

While stent 400F is illustrated without the first saddle interaction 420 or the second saddle interaction 422 as described above, it will be recognized that any combination of the embodiments described herein may be made. Various embodiments may include differently configured first and second ends. For example, a stent may include a first end with a doubled back configuration including a retention member as described with respect to one of the above embodiments, and the stent may include a second end with a configuration according to an available embodiment. In another example, a stent may include a first end with a configuration according to an available embodiment and a second end with a doubled back configuration including a retention member as described with respect to one of the above embodiments. In yet another example, a stent may include a first end and a second end, wherein each of the first end and the second end comprises a different doubled back configuration of the embodiments described above. Various embodiments may include asymmetrical configurations in order to accommodate stent engagement with various tissue types, dimensions, densities, muscular masses, drainage needs, etc.

Embodiments may include one or more additional features for preventing stent migration, regulating the flow of material through a stent, or other useful purpose. For example, a stent may include a valve disposed in a lumen of the stent to regulate fluid flow; e.g., with respect to FIG. 4G, a stent 400G may include a valve 460 disposed in the lumen 212 to regulate fluid flow through the lumen 212. A valve may include a slit valve, a valve involving one or more flaps, a duckbill valve, a funnel-shaped valve, or other valve for regulating unidirectional and/or bidirectional flow of fluid and/or particulates. A valve may include one or more wires, polymer sheets, silicone components, other materials, or any combination thereof. A valve may be placed on a first end, a second end, or anywhere along a central portion of a stent.

Additionally, or alternatively, a stent, such as stent 400G, may include one or more additional retention features, such as prongs. Prongs, such as prongs 462, may be configured to engage with a tissue in order to resist migration and/or hold the tissue in place. Prongs may be placed anywhere along a surface of a stent in order to engage with a tissue.

A cover, such as cover 220, may be applied to all or part of stent 400G, as described above with respect to FIG. 2B.

Embodiments may include at least one alternative end configuration such as a flare 464. For example, a stent 400G with a single retention member created with a doubled back end of an elongated body, such as first flange 214, and with a second end including the flare 464 may be useful in applications such as hepatic gastric stenting (HGS).

Embodiments may include multiple types of stent ends. For example, with respect to FIG. 4H, a stent 400H may include a doubled back flange configuration on one end, such as first flange 214, and a flange formed by an alternative method, such as second flange 478. A flange, such as second flange 478, may be formed by forming the elongated body 200A with a braided filament around a central mandrel 302 and around an exterior of a flange forming washer, such as second flange forming washer 308 in a single direction. A cover may be applied to all or part of stent 400H, such as cover 220 as described above with respect to FIG. 2B.

FIG. 5 illustrates a cross section of a stent 500 of the present disclosure. Various components of the stent 500 may be similar to components described above, including at least one retention member, a cylindrical saddle region, and a lumen. For example, the stent 500 may comprise one or more of first flange 214, second flange 216, cylindrical saddle region 218, and lumen 212 as described with respect to FIGS. 2A-B. The stent 500 may be positioned within a patient such that the planar surface 512 of the first retention member contacts a tissue wall 506 and the planar surface 514 of the second retention member contacts a tissue wall 508, with the cylindrical saddle region 218 extending therebetween. The tissue wall 506 and the tissue wall 508 may be walls of the same or of different tissues. For example, the first flange 214 and the second flange 216 may contact the tissue wall 506 of a first tissue and the tissue wall 508 of a second tissue so that the first tissue and the second tissue interact at tissue interface 520. In some embodiments, at least one additional tissue may be positioned between a tissue of the first tissue wall 506 and a tissue of the second tissue wall 508 (not illustrated). A lumen extending through the first retention member, second retention member, and cylindrical saddle region may therefore provide an open interior passage between a first body lumen 502 and a second body lumen 504. The stent 500 may be partially or fully covered by a cover, such as cover 220. A flat side of a retention member of the stent 500, such as flat covered side 406, may interact with an adjacent tissue 510 with lower propensity for creating abrasions than a corresponding end of a stent with one or more terminations, such as the stent end 124 as described with respect to FIG. 1A.

In use and by way of example, the stent may be disposed in a constrained configuration between an inner member and an outer sheath of a tissue-penetrating element. For example, in a constrained configuration, one or more of a first retention member, a second retention member, or a cylindrical saddle region may be restricted to a smaller outer diameter. A sharpened second end of the tissue penetrating element may be advanced through a tissue wall and into the second body lumen 504. Additionally, or alternatively, the tissue penetrating element may comprise an electrically conductive tip for advancing through a tissue wall. The second portion 516 of the stent 500 may then be advanced distally beyond the lumen of the tissue-penetrating element such that the second retention member is deployed within the second body lumen 504 and the planar surface 514 placed in contact with the tissue wall 508. The tissue-penetrating element may then be proximally retracted a predetermined distance, such as may be measured with one or more visual and/or imagable deployment markers, and the first portion 518 may be unconstrained and/or deployed in the first body lumen 502 such that the planar surface 512 contacts the tissue wall 506.

Alternatively, in the method above, a separate instrument with a sharpened distal tip may be advanced along the path above and into the second body lumen 504 to create a path, a guidewire put in place and the separate instrument withdrawn over the guidewire, and a stent, according to the various embodiments described above, loaded on a delivery catheter inserted over the guidewire, and the stent then deployed according to the steps outlined above.

Embodiments described herein may comprise various dimensions suitable for a variety of purposes and/or uses with respect to various tissues. For example, a stent comprising a wire braid may comprise a length of a saddle region of 5-40 mm in a deployed configuration.

In some embodiments, the elongate body of a stent in the constrained configuration, such as elongate body 200A, may comprise a diameter in the range inclusive of 2-6 mm, or 3-5 mm. For example, a stent may have a diameter in a constrained configuration of 10 Fr or 3.5 mm. In some embodiments, the elongate body in the constrained configuration may have a length within the range inclusive of 40-150 mm. For example, a stent in a constrained configuration may have a length within the range inclusive of 40-100 mm. In some embodiments, a stent in the constrained configuration may have a length within the range inclusive of 40-80 mm.

The cylindrical saddle region may comprise a length and a diameter. In embodiments, such as stents having double-walled flanges as first and second retention members, the length of the cylindrical saddle region may be measured as (i) the length along the elongate body between the beginning of the inward walls of each of the flanges, or (ii) the length along the body that is the shortest distance between flanges at any points along the inward walls when the stent is expanded, but not deployed in tissue, or (iii) the length along the body that is the shortest distance between flanges at any points along the inward walls when the stent is expanded and deployed in tissue. In many embodiments, a cylindrical saddle region, including under the conditions (i)-(iii) above, may have a length within the range inclusive of 5-150 mm in the expanded configuration, although in some instances the cylindrical saddle region (such as cylindrical saddle region 218 of FIGS. 2-5) may have a length in the range inclusive of 5-40 mm or 5-20 mm in the expanded configuration. Exemplary lengths of the cylindrical saddle region of devices for gastrointestinal stenting or drainage in the expanded configuration may include lengths within a range inclusive of 10-30 mm, 10-20 mm, 10-15 mm, or 5-10 mm.

In many embodiments, a diameter of the cylindrical saddle region in the expanded configuration may be greater than a diameter of the elongate body in the constrained configuration. For example, a diameter of the cylindrical saddle region (such as cylindrical saddle region 218 of FIGS. 2-5) in the expanded configuration may be within the range inclusive of 3-40 mm. In some embodiments, the diameter of a cylindrical saddle region in the expanded configuration may be within inclusive ranges of 5-25 mm, 5-20 mm, or 10-20 mm. In various embodiments, the diameter of the cylindrical saddle region in the expanded configuration may be 3-5 times greater than a corresponding diameter of the stent in the constrained configuration, although in several embodiments, the diameter of the cylindrical saddle region in the expanded configuration may be 3-10 times greater than the corresponding diameter of the stent in the constrained configuration.

A lumen of a cylindrical saddle region, in an expanded configuration, such as lumen 212 as described with respect to FIGS. 2-5, may include, for example, an inner diameter of 3-25 mm.

In embodiments, a diameter of a first retention member, a second retention member, or both in the expanded configuration may be larger than a diameter of an elongate body. For example, a diameter of the first retention member and/or a second retention member (such as first flange 214 and second flange 216 as described with respect to FIGS. 2-5) may be within the range inclusive of 5-40 mm. Other exemplary retention member diameters may be within a range inclusive of 15-40 mm or 15-35 mm. In many embodiments, a diameter of a retention member may be set to have a particular offset from a diameter of the cylindrical saddle region in the expanded configuration. For example, stents may be configured to include a 3-20 mm difference between a diameter of the cylindrical saddle region and a larger diameter of the first retention member and/or a second retention member in the expanded configuration. Some embodiments may include a 6-10 mm difference between an outer diameter of the cylindrical saddle region and a larger outer diameter of a retention member. For example, for an outer diameter of a cylindrical saddle region of 10 mm, an outer diameter of a first flange and/or a second flange may be between 16-20 mm. It will be understood that some embodiments may include greater differences between an outer diameter of a cylindrical saddle region and an outer diameter of a first flange and/or a second flange. In some embodiments, such as in esophageal stents, a difference between an outer diameter of a cylindrical saddle region and an outer diameter of a first flange and/or a second flange may be 4-6 mm. Other embodiments may comprise larger and/or smaller differences between an outer diameter of a cylindrical saddle region and an outer diameter of a first flange and/or a second flange. In other examples, a retention member may be configured to have a diameter that is 1-5 times greater than the diameter of the cylindrical saddle region in the expanded configuration. For example, for a device with the cylindrical saddle region having a diameter of 10 mm in an expanded configuration, the first retention member and/or a second retention member may have a diameter within inclusive ranges of 13-30 mm, 15-25 mm, or 16-20 mm. In another example, a stent with the cylindrical saddle region in the expanded configuration having a diameter of 20 mm may have one or more retention members with a diameter within the inclusive range of 23-40 mm. It will be understood that some embodiments may include greater or lesser offsets between the diameter of the cylindrical saddle region and the larger diameter of the first retention member and/or the second retention member.

Referring to FIGS. 2-5, a first retention member, such as first flange 214, may have a same or different axial width as a second retention member, such as second flange 216, wherein the axial width of a retention member may be measured as a distance along the longitudinal axis between an inward wall and outward wall of a respective retention member. In some embodiments, the width of the first retention member and/or the second retention member in the expanded configuration may be within the inclusive range of 0.5-10.0 mm. In some embodiments, a width of a first flange and/or a width of a second flange may be within the inclusive range of 0.5-3.0 mm. Other embodiments may include a smaller and/or greater width of the first retention member and/or the second retention member, such as within the inclusive rages of 0.5-6 mm, 2-6 mm, or 3-7 mm. In some embodiments, a retention member may have a constant width. In other embodiments, a width of a retention member may vary along a vertical plane.

In some embodiments, a first protruding end and/or second protruding end, such as protruding ends 232, 234 as described with respect to FIGS. 2-5 may have a length within the range inclusive of 0-3 mm in the expanded configuration, such as within the inclusive ranges of 0.5-2.5 mm or 1.0-1.5 mm. The first protruding end and/or second protruding end may define a lumen, such as a flange lumen contiguous with the cylindrical saddle region, with a diameter greater than or equal to the diameter of the lumen defined by the cylindrical saddle region, or saddle lumen. In various embodiments, a lip, or projection, may comprise a flange lumen diameter in the expanded configuration that is at least 0.5-2.0 mm wider than the diameter of a corresponding cylindrical saddle region in an expanded configuration, for example, within the diameter range inclusive of 3.5-42 mm for a saddle diameter within the range inclusive of 3-40 mm. As further exemplary inclusive ranges, for a corresponding cylindrical saddle region diameter of 10 mm in the expanded configuration, a lip may have a diameter of 11-14 mm. For a corresponding cylindrical saddle region diameter of 15 mm in the expanded configuration, a lip may have a diameter of 16-19 mm. In another example, a cylindrical saddle region diameter of 20 mm in the expanded configuration may correspond with a lip diameter of 21-24 mm. A lip may be formed contiguously with the retention members and/or with the saddle lumen. The lip may be parallel to the longitudinal axis of the cylindrical saddle region. Alternatively, or additionally, the lip may be non-parallel to the longitudinal axis. For example, the lip may extend radially outward or inward along the longitudinal axis. A radially outward extension of the lip may encourage flow through a lumen thereof, for example, by acting as a funnel into the lumen. A radially inward extension of the lip may discourage flow through a lumen thereof, for example, by defining a smaller entrance thereof.

Various embodiments may include total stent lengths ranging from 5-60 mm in the expanded configuration. For example, exemplary deployed stents may have lengths of 10-50 mm or 10-40 mm.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. A stent, comprising:
an elongate body;
a first flange, a second flange, and a central portion therebetween formed from the elongate body in an expanded configuration;
wherein ends of the elongate body are doubled back into the respective first and second flanges.

2. The stent of claim 1, wherein the elongate body is formed across an inner opening of the first and second flanges.

3. The stent of claim 1, wherein the stent comprises an unexpanded configuration when constrained by a delivery device prior to being deployed.

4. The stent of claim 3, wherein a diameter of the first flange, the second flange, or both is larger than a diameter of the elongate body in the expanded configuration.

5. The stent of claim 1, wherein the ends are doubled back entirely into the respective first and second flanges such that respective ends of the stent comprise flush end faces.

6. The stent of claim 1, wherein the doubled back ends of the elongate body extend beyond one or both of the respective first and second flanges along the elongate body towards the opposing flange.

7. The stent of claim 1, further comprising a coating.

8. The stent of claim 7, wherein the coating constrains at least one doubled back end of the elongate body along a radially outward surface of the elongate body.

9. The stent of claim 1, wherein the elongate body is self-expanding and comprises a shape-memory material.

10. A method of forming a stent, comprising:
forming an elongate body around a central mandrel, the elongate body comprising a first portion, a second portion, and a cylindrical saddle region extending therebetween in an expanded configuration; and
forming at least one retention member by one or both of:
doubling back the first portion over a first flange forming washer to form a first retention member, and
doubling back the second portion over a second flange forming washer to form a second retention member.

11. The method of claim 10, wherein the elongate body comprises a shape-memory material, and further comprising heat setting the expanded configuration for the stent having the at least one retention member.

12. The method of claim 10, further comprising covering at least part of the elongate body with a coating.

13. The method of claim 12, wherein the coating constrains a first termination of the first portion, or the coating constrains a second termination of the second portion, or both.

14. A stent, comprising:
a stent body having a constrained configuration and an expanded configuration, the stent body comprising a tubular structure including a first portion, a second portion, and a central portion extending therebetween in the expanded configuration, wherein one or both of the first portion and the second portion are doubled back toward the opposing portion to form, respectively, a first flange and second flange.

15. The stent according to claim 14, wherein the stent body is formed across an inner opening of the first and second flanges.

16. The stent of claim 14, wherein the doubled back ends of the tubular structure extend beyond one or both of the respective first and second flanges along the tubular structure towards the opposing flange.

17. The stent of claim 14, wherein the ends are doubled back entirely into the respective first and second flanges such that respective ends of the stent comprise flush end faces.

18. The stent of claim 14, wherein the doubled back ends of the tubular structure extend beyond one or both of the respective first and second flanges along the tubular structure towards the opposing flange.

19. The stent of claim 14, further comprising a coating.

20. The stent of claim 15, wherein the coating constrains at least one doubled back end of the tubular structure along a radially outward surface of the tubular structure.
